(19) United States
(12) Patent Application Publication
(10) Pub. No.: US 2003/0077571 A1
(43) Pub. Date: Apr. 24, 2003

(54) Experimental model and method for evaluation of therapeutic agents against asthenopia

(76) Inventor: Iwao Katsuyama, Osaka (JP)

Correspondence Address: BROWN & MICHAELS, PC, 400 M & T BANK BUILDING, 118 NORTH TIOGA ST, ITHACA, NY, 14850, US

(21) Appl. No.: 10/092,210

(22) Filed: Mar. 6, 2002

(30) Foreign Application Priority Data
Apr. 26, 2001 (JP) 2001-130414
Feb. 26, 2002 (JP) 2002-50116

(51) Int. Cl. C12Q 1/00; C12N 5/06

(52) U.S. Cl. 435/4; 435/325

(57) ABSTRACT

This invention provides a experimental model and method for evaluating and screening drugs or quasi-drugs against asthenopia, including accommodative asthenopia, by preparing a ciliary muscle sample enucleated from a non-human animal, inducing contraction by one or more of contraction stimulants, measuring the contraction rate of the ciliary muscle, treating it with a test formulation and measuring the contraction rate of the ciliary muscle after such treatment.

### REFERENCE TO RELATED APPLICATIONS

This application claims priority of Japanese Patent Application No. 2002-50116, filed Feb. 26, 2002, and Japanese Patent Application No. 2001-130414, filed Apr. 26, 2001. The benefit under 35 USC §§ 119 (a-d) of the foregoing Japanese Patent Applications is hereby claimed, and the aforementioned applications are hereby incorporated herein by reference.

### BACKGROUND OF THE INVENTION

1. Field of the Invention

The invention pertains to the field of experimental models useful as an assay system for the evaluation of the therapeutic effects of medicines against asthenopia. More particularly, the invention pertains to the use of this model in an in vitro method for screening potential medicines for the treatment of patients having asthenopia and accommodative asthenopia.

2. Description of Related Art

The number of patients suffering asthenopia is steadily increasing due to a growing use of office automation equipment in the work place. Usually only eye drops have been used to treat asthenopia. However, the therapeutic effects of such eye drops against asthenopia are unsatisfactory, due in part to the lack of appropriate preclinical assay models and test methods for evaluating and screening eye drop medications.

Ciliary muscle is a smooth muscle which makes up the major segment of the ciliary body surrounding the eye lens in ocular tissues. Asthenopia, in particular accommodative asthenopia, is defined as a difficulty in eye lens adjustment associated with fatiguing of the ciliary muscle; Journal of Japanese Ophthalmology Association vol. 92, 1854-1858(1988). The ciliary muscle, when stimulated, undergoes isometric contraction, i.e., it contracts without a change in length of the muscle itself, and fatiguing of the ciliary muscle is reflected by declining tension during muscular contraction and a delay in the contraction/relaxation process. Therefore, one of the appropriate methods for evaluating medicines, such as, eye drops, against asthenopia is to use an enucleated organ (biological sample). For example, one method of the so-called “Magnus” type directly observes the change in tension of muscular contraction and delay of the contraction/relaxation process using ciliary muscle suspended in a Magnus tube.

Magnus methods are usually carried out by use of a Magnus apparatus which is mainly comprised of: (1) a Magnus tube of about 1-2 cm in diameter and about 2-5 cm in depth for the immersion of a biological organ sample in an artificial nutrient solution, (2) an air ventilation tube for the supply of air to the nutrient solution, (3) an anchor to for fixing one end of the organ sample, (4) a tension transducer for fixing the another end of the biological organ sample and measuring the tension thereof, and (5) a thermostat to keep the temperature of the Magnus tube at a constant level (e.g., 37 degrees C.). Magnus methods are advantageous for measuring muscular contraction under physiological conditions, because the enucleated organ is free from the various influences of other organs and yet it does not loose its mobility due to the nerve plexus remaining in the muscle.

Known Magnus methods include the study of the M3-type muscarinic receptor in the ciliary muscle of the cow (Hiroshi Matsuda et al: Gen. Pharmac., 30 (4), 579-584 (1998)), and the study of the relaxation response to nitrous oxide in the ciliary muscle of the cow (Soichiro Kamiawa et al: Exp. Eye Res., 66, 1-7 (1998)), (Hiroshi Masuda et al: Current Eye Res., 16, (12), 1245-1251, (1997)). Alternatively, the Magnus system can be used to evaluate the relaxation effect of eye drops on the contraction of ciliary muscle induced by endothelin-1 (Japan patent publication No. H09-59173), or by potassium chloride or carbachol (Japan patent publication No. H07-133225). However, these KNOWN magnus methods evaluate only the preventive effects of medicines against transient contraction of ciliary muscle induced by endotherin-1, potassium chloride or Carbachol, these making the use of said particular chemical compounds questionable in magnus methods which are carried out in vitro. In other words, in vitro a Magnus method combined with the use of the above compounds does not necessarily replicate the fatiguing of ciliary muscle that occurs in asthenopia in vivo.

### SUMMARY OF THE INVENTION

In view of the problems associated with current Magnus methods and the absence of a suitable in vitro model of asthenopia, the present invention provides a new approach, as described herein. Thus, an object of the present invention is to provide a novel experimental model that is useful as an assay system for the evaluation of the therapeutic effects of medicines against asthenopia. Another object of the invention is to provide a novel test method that can be used to screen and evaluate medicines for the treatment of asthenopia. Still another object of the invention is to provide the experimental model and an in vitro test method suitable for screening and evaluating medicines that are effective against accommodative asthenopia. As described herein, this invention makes it possible to replicate in vitro the asthenopia which occurs in vivo. Further objects of the invention are to provide an experimental model and test method for evaluating the therapeutic effects of medicines on asthenopia quantitatively, for example, in terms of ED50, and the like. Further objects, features and advantages of the present invention will be apparent from the following detailed description. It should be understood, however, that the detailed description and examples given below, while identifying preferred embodiments, should not be considered limiting to these, as various changes and modifications within the spirit and scope of the invention will be apparent to persons skilled in the art from the description.

### BRIEF DESCRIPTION OF THE DRAWING

FIG. 1 shows a preliminary test for measuring the contraction rate of ciliary muscle in vitro upon stimulation by acetylcholine. Contraction of ciliary muscle was induced by the addition of acetylcholine, and the contraction rate was determined as a function of the number of stimulations. Before the tenth stimulation, varying concentrations of cyanocobamin were added.

FIG. 2 shows a graph showing a comparison of the contraction rate of ciliary muscle for different doses of cyanocobamin in a preliminary test.

FIG. 3 shows a graph showing changes in contraction rate of ciliary muscle as a function of stimulation by acetylcholine in a final test. At the tenth stimulation, varying concentrations of cyanocobalamin were added.

FIG. 4 shows a graph showing a comparison of the contraction rate of ciliary muscle for different doses of cyanocobalamin in a final test.

### DETAILED DESCRIPTION OF THE INVENTION

According to the one aspect of the present invention, there is provided an experimental model of asthenopia, wherein said asthenopia is caused by inducing repeated contractions of the ciliary muscle derived from a non-human test animal until said ciliary muscle exhibits a substantially stable decrease in the tension of muscular contraction.

According to another aspect of the invention, there is provided an experimental model of asthenopia as described above in which the asthenopia is accommodative asthenopia.

According to still another aspect of the invention, there is provided an experimental model of asthenopia in which the ciliary muscle is enucleated from a non-human mammalian animal or from a fowl.

According to a further aspect of the invention, there is provided an experimental model of asthenopia in which the ciliary muscle is contracted a plurality of times by the use of smooth muscle contraction-inducing means.

According to still another aspect of the invention, there is provided an experimental model of asthenopia wherein the smooth muscle contraction-inducing means is a chemical stimulant, preferably through not necessarily selected from the group consisting of acetylcholine, serotonin, histamine, muscarine, nicotine and endothelin.

According to another aspect of the invention, there is provided an experimental model of asthenopia wherein the smooth muscle contraction-inducing means is an electrical stimulant.

According to a further aspect of the invention, there is provided an experimental model of asthenopia, such as described above, wherein the contraction of ciliary muscle is repeated at least three times to give a substantially stable decrease in the tension of muscular contraction.

According to the further aspects of the invention, there is provided an experimental model of asthenopia as described above wherein the ciliary muscle shows a decrease of 50±30% in the tension of muscular contraction, or decrease of 50±20% in the tension of muscular contraction, or a decrease of 50±10% in the tension of muscular contraction.

According to still another aspect of the invention, there is provided a method of preparing an experimental model of asthenopia, which comprises the step of inducing repeated contractions in vitro of ciliary muscle taken from a non-human test animal until the ciliary muscle exhibits a substantially stable decrease in the tension of muscular contraction.

According to still another aspect of the invention, there is provided a method for evaluating a therapeutic agent against asthenopia comprising the steps of contacting the ciliary muscle from a non-human animal with said agent, and measuring the effect on the contraction of said ciliary muscle.

The effect of the agent can be evaluated by comparing the decrease in tension of muscular contraction before and after contact with the agent.

This evaluation can be carried out by the use of a Magnus apparatus.

The ciliary muscle to be used in this in vitro model of asthenopia is obtained by enucleation from non-human test animals. Examples of such animals include various non-human mammalian species and fowls such as cows, sheep, pigs, goats, monkeys, dogs, cats, rabbits, guinea pigs, rats, mice, hens, cocks, quail and ostriches. Mammals are preferred.

The animals, before being subjected to enucleation of the ciliary muscle, are preferably bred under well-controlled conditions to obtain consistent preparations of the ciliary muscle. After enucleation, the ciliary muscle may preferably be cut to an appropriate size in order to obtain a biological sample. The size of the organ may be chosen as desired. For example, a suitable size for a Magnus method is preferably about 3 mm wide and about 6 mm long.

Contraction of the enucleated ciliary muscle should be done a plurality of times in order to give a substantially stable decrease in the tension of the muscular contraction. Any smooth muscle contraction-inducing means, i.e., any stimuli to induce the reversible contraction of smooth muscle, may be used for this purpose. For example, the contraction may be induced either by physical stimuli, chemical stimuli, or electrical stimuli. Suitable examples of chemical stimuli include acetylcholine, serotonin, histamine, muscarine, nicotine, endothelin, and the like. These stimuli may be used either individually or in any combination. Among them, acetylcholine is preferably used, since it is a naturally occurring stimulant which stimulates the cholinergic receptor to induce smooth muscle contraction, being liberated from the parasympathetic nerve.

Serotonin is an effective intracerebral chemical transmitter and normally exists in the intestinal chromaffin cells of the intestinal mucosa. It moves into blood platelets from the chromaffin cells and induces contraction in vascular smooth muscle or in smooth muscles such as enucleated intestinal canal and muscularis of the bronchus during pulmonary circulation.

Histamine is stored in basophils in the blood and in tissue mast cells and plays a leading role in inflammation and allergy. It causes the accentuation of intestinal peristalsis and gastric acid secretion, and the dilation, accentuation and permeability accentuation of minute capillary blood vessels. It also induces a strong contraction of bronchus and smooth muscle, e.g., in blood vessels.

Muscarine is an alkaloid of the toadstool origin and, like acetylcholine, stimulates the cholinergic receptor, which can be blocked by atropine. It acts on postsynaptic membranes and induces the strong contraction of bronchus and smooth muscle, e.g., in blood vessels.

Nicotine is an alkaloid obtained from tobacco leaves, like acetylcholine, and stimulates cholinergic receptors, which can be blocked by hexamethonium. It acts on the neuromuscular synapses of autonomic ganglia and motor end plates, and induces a strong contraction of bronchus and smooth muscle, e.g., in blood vessels. Because of these activities, nicotine is preferred to use in this invention.

Endothelin is also preferred as a chemical stimulant because endothelin, a polypeptide of 21 amino acids produced by human or pig epithelial cells, induces the strong contraction of blood vessels and manifests a long-lasting hypertensive activity.

While the above stimulants are different from each other in their mechanisms of action, they all induce contraction in the smooth muscle of the bronchus, blood vessels and the intestinal canal. They also induce the contraction of smooth ciliary muscle. The concentration of the chemical stimulants to be used in the practice of this invention may be selected based on the stimulants used and/or purpose of the experiments. It is usually preferred to use them at concentrations of from 10-10 to 10-1 mol/L, and more preferably from 10-9 to 10-2 mol/L.

When electrical stimuli are used for preparing the experimental model of the invention, the degree of stimulation may be changed or controlled by various conditions such as the manner of electric loading (direct or indirect stimulation), type of electric current (alternate or direct), or the power of electric current or voltage, and/or by the period, intervals or frequency of electrical stimulation. The degree of stimulation may also vary, depending on other factors such as an apparatus to be used, the shape and materials of the electrode, the size of the biological sample, the positional relationship between the biological sample and electrode, or the materials which lie between the biological sample and electrode. For the present invention, a suitable combination of these conditions may be readily decided based on the degree of ciliary muscle contraction to be obtained, for example, making reference to the conditions described in Masuda et al's Gen. Pharmac. 30, 579-584(1998), the contents of which are hereby incorporated by reference.

It is recommended, although not absolutely required, that a biological sample for contraction experiments be suspended in a conventional tube such as a Magnus tube. The enucleated organ, which is free from the control of central nerves, does not loose its mobility because of the nerve plexus which remains in the muscle tissue. When the experiment is carried out by a Magnus method, the Magnus tube is filled with a conventional nutrient solution such as Krebs-Henseleit solution, and the tube is preferably bubbled through with a mixture of carbon dioxide and oxygen (e.g., 5% carbon dioxide and 95% oxygen). Multiple stimulations can be performed on the same sample after recovery from a previous contraction.

In practice, a sample of ciliary muscle as described above is suspended in the Magnus tube with a loading weight using a tensile transducer, and the first stimulation is preferably given thirty minutes after suspension to induce muscle contraction. In treatment a chemical stimulant, such as atropine, is used as the smooth muscle contraction-inducing means, it is preferred that the stimulant be added to the nutrient solution so that the final concentration of the stimulant at the first contraction is about 10-4 mol/L. When the first contraction reaches a plateau, the sample is washed until the transducer signal returns to the baseline. Contraction can be repeated by the addition of the same stimulant. The number of stimulations can be chosen ad libitum, depending on the purposes of the experiments. The preferred number of stimulations is 3 to 50 times, preferably 3 to 20 times, and most preferably 4 to 15 times. A high level fatiguing of the ciliary muscle may be achieved by such repetitive stimulations, but stimulation of more than 50 times may give no substantially additional benefit. Usually, repeating contractions by use of a moderate stimulation is preferred to obtain a stable decrease in the tension of muscular contraction.

It is preferred that the change in transducer signal as a measure of contraction is recorded after each stimulation.

The experimental data thus obtained may be expressed in any known manner. A convenient method for recording the data is as follows. The tension of the sample is measured after each stimulation. The baseline tension is subtracted from the tension for each stimulation to obtain a net tension. The first net tension, i.e., the tension after the first stimulation—baseline tension, is set as 100% and any subsequent net tension is divided by the first net tension.

It is preferred that the ciliary muscle sample which, after repeated stimulations thereof, shows a stable decrease of 50±30%, more preferably 50±20% decrease, and most preferably 50±10% decrease, in the tension of muscular contraction is used as a standard sample for subsequent experiments.

When electrical stimuli are used instead of chemical stimuli, it is preferred that the biological organ sample showing a stable decrease of 50±30%, more preferably 50±20% decrease, and most preferably 50±10% decrease, in tension of muscular contraction is used as a standard sample for subsequent experiments using the Magnus apparatus.

After the above-mentioned repeated stimulations, the therapeutic effectiveness of a medicine can be readily evaluated in conventional manner, i.e., by treating or contacting the sample with said medicine. For example, said treatment or contact may preferably be carried out by replacing the solution in the Magnus tube with a nutrient solution(e.g., Krebs-Henseleit solution) containing the medicine being tested. Then, stimulation is applied, from which the contraction rate is determined relative to the first contraction. If required, the sample may also be pretreated with atropine, preferably at a final concentration of 10-6 mol/L, in order to confirm whether or not the sample responds to contract by the contraction stimulant.

The above experiment may be carried out as a preliminary optimization for a test medicine and the results compared with other available medicines or formulations.

The term “medicine” as used herein includes any compound, material, composition or formulation which is prepared and/or used for screening and/or evaluating its therapeutic effect on asthenopia. It includes within its scope various drugs , quasi drugs, functional foods or food supplements, and should be construed in a broad sense and not be restricted to those which have already been made available in the marketplace. Potential drugs and so forth, i.e., those which are prepared and/or used for research and/or development purposes, are also included within the scope of the term “medicine” as used herein. For the practice of this invention, these medicines may be used in the form of solution, emulsion, suspension, ointment, injection, eye drop, or the like. Examples of existing medicines that can be evaluated by the present invention include the formulation of vitamin B12 (cyanocobalamin) available under the trade names: Sancoba eye drop 0.02% (made by Santen Pharmaceutical Co., Ltd.), PTO-Q05 (made by TOA Pharmaceuticals Co., Ltd.), Cabalam eye drop 0.02% (made by Nippon Tenganyaku), Softar eye drop 0.02% (made by Senju Pharmaceutical Co., Ltd.), TP263 (0.02%, made by Toyo Pharma Kabushiki Kaisha); and the formulation of flavine adenine dinucleotide available under the trade names: FAD eye drop 0.05% (made by Santen Pharmaceutical Co., Ltd.), FAD T eye drop 0.05% (made by Nippon Tenganyaku), Vitast eye drop 0,05% and 0.1% (made by Senju Pharmaceutical Co., Ltd. and Takeda Chemical Industries, Ltd), Flavitan eye drop 0.05% (made by TOA EIYO Pharmaceutical Company and Yamanouchi Pharmaceutical Co., Ltd.).

Since the same sample of ciliary muscle can be used to test multiple medicines, it is possible to compare various drugs without influence by specimen-to-specimen variations.

### DESCRIPTION OF SPECIFIC EMBODIMENTS

The following examples are provided merely to illustrate the invention, and are not to be interpreted as limiting the scope of the invention to these which are described in the specification and defined in the claims.

### EXAMPLE 1

### Preparation of Sample

Eight rabbits were used after quarantine and conformation. Breeding condition were as follows: the rabbits were bred individually in an animal rearing box placed indoors at a room temperature of 21±3 degrees C., relative humidity of 50±20%, using an illumination time of 12 hours (7 a.m. light-up, 7 p.m. light-out) and ventilation of 10-15 times per hour.

The test animals were put under general anesthesia by intramuscular injection of a mixed solution of Ketalar: Seraktal (4:5) at 1 mL/kg followed by enucleation of the eyeballs. In more detail, the anesthetic agent used was; ketamine hydrochloride parenteral solution (made by Sankyo Co., Ltd., specifically Ketalar 50 for intramuscular injection) and xylidine hydrochloride parenteral solution (made by Beyer Yakuhin, Ltd., specifically Seraktal 2% for injections of dogs and cats).

Each enucleated eyeball was sclerotomized and amputated to half at the equatorial position, and then the ciliary muscle was carefully ablated from the sclera after removal of the crystalline lens. The ciliary muscle thus obtained was cut into a strip of 3 mm in width and 6 mm in length and used in the protocols described below. For the above surgery, conventional ophthalmic surgical instruments such as a keratotome, a micro knife, a pincette and scissors were used.

### EXAMPLE 2

### Generation of Fatiguing

The ciliary muscle obtained in EXAMPLE 1 was suspended in a Magnus tube filled with Krebs-Henseleit solution bubbled through with a mixed gas of 5% carbon dioxide and 95% oxygen. Krebs-Henseleit solution was prepared as follows. A-solution, B-solution, C-solution, water and D-solution were mixed at a ratio of 1:1:1:6:1 in the following order. First A-solution, B-solution and C-solution were mixed in the above ratio followed by the addition of distilled water. After rigorous mixing, D-solution was added. The solution thus prepared was shielded from light and used after heating at 37 degrees C.

The preparation of A- to D-solutions was as follows. A-solution: distilled water was added to 69.2 grams of NaCl, 3.50 grams of KCl, 1.63 grams of KH2PO4, 2.96 grams of MgSO47H2O for a final volume of 1000 ml. B-solution: distilled water as added to 4.00 grams of glucose for a final volume of 200 ml. C-solution: distilled water was added to 4.20 grams of NaHCO3 for a final volume of 200 ml. D-solution: distilled water was added to 3.68 grams of CaCl22H2O for a final volume of 100 ml.

The sample in the Magnus tube was suspended at a loading weight of 0.4 g using a tension transducer (Isometric Transducer, FD Pick-up TB-611 T type, made by Nihon Kohden Corporation, Tokyo, Japan). After equilibration for 30 minutes, acetylcholine (made by SIGMA, St. Louis, Mo.) at a final concentration of 10-4 mol/L was added for the first stimulation. After tension reached a plateau value, the sample was washed with a buffer until the tension returned to the baseline. This procedure was repeated nine times.

The tension of the sample upon each stimulation was recorded by the data collection software (VISUAL DESIGNER ver. 2.3, made by INTELLIGENT INSTRUMENTATION, Tucson, Ariz.) via the tension transducer, an input box (JD-112S type, made by Nihon Kohden Corporation), an amplifier for pressure strain (AP-621G type, made by Nihon Kohden Corporation) and an A.D. conversion board (PCI-20428W, made by INTELLIGENT INSTRUMENTATION).

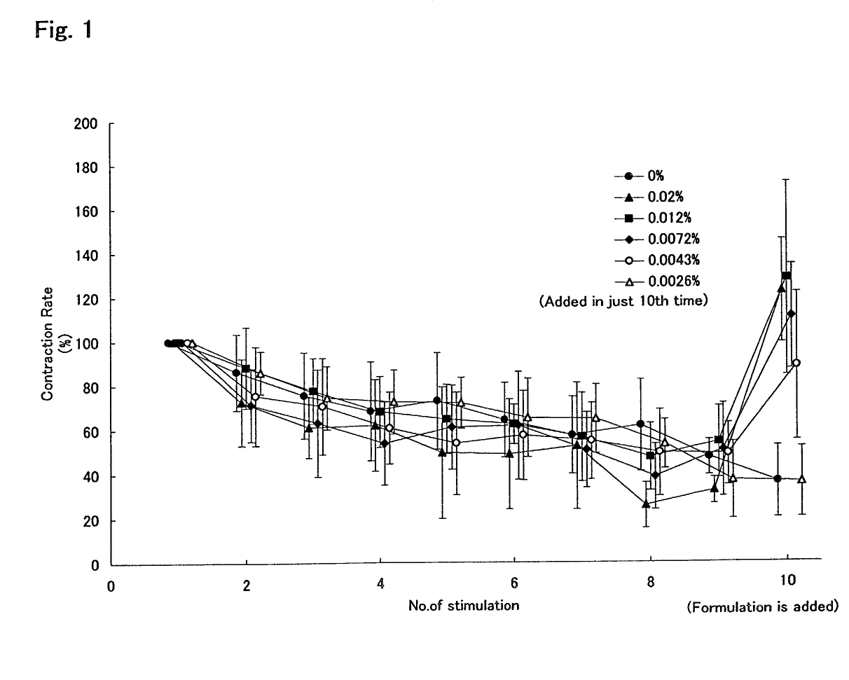

The net tension was calculated by subtracting the baseline tension from the tension observed after stimulation, and this was defined as the contraction rate. The first net tension, i.e., tension at the first stimulation—baseline tension, was set as a contraction rate of 100%. The results are shown in Tables 1-5 and in FIG. 1.

In addition, the sample in the Magnus tube was treated with atropine at a final concentration of 10-6 mol/L and subjected to acetylcholine stimulation as described above, in order to confirm whether the tension of the sample was in fact due to stimulation by acetylcholine. The results are shown in Tables 6-8.

| No. of | | | | | | |
|---|---|---|---|---|---|---|
| measureing | Average of Contraction Rate (%) ± standard deviation | | | | | |
| 2nd | 86.4 ± 17.3 | 72.7 ± 19.7 | 88.4 ± 18.3 | 71.5 ± 16.6 | 75.6 ± 22.4 | 86.2 ± 9.6 |
| | — | N.S. | N.S. | N.S. | N.S. | N.S. |
| 3rd | 75.8 ± 19.5 | 61.3 ± 13.7 | 77.8 ± 14.8 | 63.3 ± 24.3 | 70.9 ± 21.7 | 74.6 ± 14.3 |
| | — | N.S. | N.S. | N.S. | N.S. | N.S. |
| 4th | 68.5 ± 22.4 | 62.1 ± 20.7 | 68.2 ± 16.0 | 53.9 ± 18.8 | 60.8 ± 16.3 | 72.6 ± 14.5 |
| | — | N.S. | N.S. | N.S. | N.S | N.S. |
| 5th | 73.0 ± 21.8 | 49.6 ± 29.7 | 64.8 ± 15.6 | 61.1 ± 19.1 | 53.8 ± 23.2 | 72.2 ± 11.5 |
| | — | N.S. | N.S. | N.S. | N.S. | N.S. |
| 6th | 64.4 ± 16.9 | 49.0 ± 24.9 | 62.5 ± 9.0 | 61.9 ± 24.4 | 57.5 ± 20.1 | 65.4 ± 17.7 |
| | — | N.S. | N.S. | N.S. | N.S. | N.S. |
| 7th | 57.5 ± 17.4 | 52.6 ± 28.7 | 56.7 ± 20.0 | 50.9 ± 17.0 | 54.9 ± 17.2 | 64.9 ± 15.5 |
| | — | N.S. | N.S. | N.S. | N.S. | N.S. |
| 8th | 61.9 ± 20.7 | 25.7 ± 10.3 | 47.6 ± 15.2 | 38.7 ± 14.9 | 49.6 ± 19.6 | 53.5 ± 11.0 |
| | — | * | N.S. | N.S. | N.S. | N.S. |
| 9th | 47.5 ± 7.9 | 32.5 ± 6.0 | 54.4 ± 16.1 | 50.8 ± 21.0 | 48.9 ± 14.1 | 37.0 ± 17.5 |
| | — | N.S. | N.S. | N.S. | N.S. | N.S. |
| Cyanocobalamin | Control 0% | 0.02% | 0.012% | 0.0072% | 0.0043% | 0.0026% |
| Concentration | | | | | | |
| 10th | 36.3 ± 16.3 | 122.7 ± 23.4 | 128.3 ± 43.8 | 111.1 ± 23.7 | 88.6 ± 33.5 | 36.1 ± 16.0 |
| | — | ** | ** | ** | * | N.S. |

**P < 0.01
*P < 0.05
N.S. no significant (by Dunnett multiple comparison)

TABLE 2

| | Concen- | No. of | Enucleated Organ No./Tension of Contraction (g) | | | | | |
|---|---|---|---|---|---|---|---|---|
| Group | tration | measureing | 1 | 2 | 3 | 4 | 5 | 6 |
| cyanoco- | Control | 1st | 0.0199 | 0.0315 | 0.1726 | 0.1400 | 0.1520 | 0.1824 |
| balamin | 0% | 2nd | 0.0404 | 0.0476 | 0.1721 | 0.1273 | 0.1316 | 0.1291 |
| | | 3rd | 0.0428 | 0.0608 | 0.1609 | 0.1053 | 0.0969 | 0.1184 |
| | | 4th | 0.0355 | 0.0569 | 0.1365 | 0.1336 | 0.0916 | 0.1174 |
| | | 5th | 0.0404 | 0.0442 | 0.1316 | 0.0755 | 0.0881 | 0.0984 |
| | | 6th | 0.0106 | 0.0120 | 0.0745 | 0.0701 | 0.0813 | 0.0974 |
| | | 7th | 0.0179 | 0.0193 | 0.1287 | 0.0511 | 0.0803 | 0.0847 |
| | | 8th | 0.0159 | 0.0242 | 0.0901 | 0.0359 | 0.0671 | 0.0754 |
| | | 9th | 0.0316 | 0.0154 | 0.0911 | 0.0320 | 0.0564 | 0.0686 |
| | | 10th | 0.0174 | 0.0139 | 0.0872 | 0.0218 | 0.0476 | 0.0583 |
| | 0.02% | 1st | 0.1648 | 0.1399 | 0.0624 | 0.1135 | 0.1594 | 0.2184 |
| | | 2nd | 0.1614 | 0.1546 | 0.0638 | 0.1316 | 0.1340 | 0.1706 |
| | | 3rd | 0.1780 | 0.1433 | 0.0750 | 0.1004 | 0.1227 | 0.1721 |
| | | 4th | 0.1819 | 0.1384 | 0.0677 | 0.1106 | 0.0959 | 0.1555 |
| | | 5th | 0.1189 | 0.1370 | 0.0682 | 0.0789 | 0.0695 | 0.1306 |
| | | 6th | 0.1170 | 0.1370 | 0.0638 | 0.0823 | 0.0407 | 0.1052 |
| | | 7th | 0.1280 | 0.1038 | 0.0482 | 0.0545 | 0.0261 | 0.1013 |
| | | 8th | 0.1052 | 0.0467 | 0.0467 | 0.0462 | 0.0256 | 0.0837 |
| | | 9th | 0.0955 | 0.0477 | 0.0477 | 0.0237 | 0.0192 | 0.0773 |
| | | 10th | 0.0769 | 0.0389 | 0.0389 | 0.0227 | 0.0056 | 0.0358 |
| | 0.012% | 1st | 0.0959 | 0.0950 | 0.0540 | 0.0871 | 0.0730 | 0.1111 |
| | | 2nd | 0.0872 | 0.1199 | 0.0462 | 0.0905 | 0.0633 | 0.1121 |
| | | 3rd | 0.0872 | 0.1155 | 0.0564 | 0.0812 | 0.0535 | 0.1009 |
| | | 4th | 0.0891 | 0.0989 | 0.0642 | 0.0690 | 0.0506 | 0.1184 |
| | | 5th | 0.0530 | 0.0867 | 0.0286 | 0.0558 | 0.0271 | 0.0647 |
| | | 6th | 0.0398 | 0.0857 | 0.0266 | 0.0588 | 0.0257 | 0.0774 |
| | | 7th | 0.0354 | 0.0730 | 0.0149 | 0.0378 | 0.0393 | 0.0730 |
| | | 8th | 0.0417 | 0.0666 | 0.0208 | 0.0422 | 0.0232 | 0.0711 |
| | | 9th | 0.0315 | 0.0579 | 0.0159 | 0.0226 | 0.0183 | 0.0506 |
| | | 10th | 0.0540 | 0.0125 | 0.0140 | 0.0231 | 0.0164 | 0.0262 |
| | 0.0072% | 1st | 0.0725 | 0.0823 | 0.1086 | 0.0594 | 0.1394 | 0.1540 |
| | | 2nd | 0.0921 | 0.0779 | 0.1282 | 0.0980 | 0.0925 | 0.1458 |
| | | 3rd | 0.0413 | 0.0862 | 0.1282 | 0.0955 | 0.0823 | 0.1418 |
| | | 4th | 0.0769 | 0.0843 | 0.1145 | 0.0975 | 0.0799 | 0.1228 |
| | | 5th | 0.0286 | 0.0686 | 0.0891 | 0.0843 | 0.0706 | 0.1160 |
| | | 6th | 0.0467 | 0.0618 | 0.0823 | 0.0589 | 0.0691 | 0.1018 |
| | | 7th | 0.0384 | 0.0628 | 0.0720 | 0.0526 | 0.0667 | 0.0774 |
| | | 8th | 0.0418 | 0.0462 | 0.0735 | 0.0262 | 0.0642 | 0.0764 |
| | | 9th | 0.0159 | 0.0481 | 0.0144 | 0.0321 | 0.0481 | 0.0676 |
| | | 10th | 0.0657 | 0.0301 | 0.0115 | 0.0301 | 0.0520 | 0.0535 |
| | 0.0043% | 1st | 0.0804 | 0.1140 | 0.0667 | 0.1243 | 0.1043 | 0.0935 |
| | | 2nd | 0.0696 | 0.1150 | 0.0540 | 0.1414 | 0.0848 | 0.0935 |
| | | 3rd | 0.0301 | 0.1101 | 0.0321 | 0.0940 | 0.1053 | 0.0716 |
| | | 4th | 0.0403 | 0.1028 | 0.0384 | 0.0613 | 0.0984 | 0.0535 |
| | | 5th | 0.0369 | 0.0935 | 0.0282 | 0.0583 | 0.0789 | 0.0530 |
| | | 6th | 0.0340 | 0.0832 | 0.0260 | 0.0691 | 0.0584 | 0.0457 |
| | | 7th | 0.0350 | 0.0901 | 0.0267 | 0.0374 | 0.0467 | 0.0350 |
| | | 8th | 0.0393 | 0.0847 | 0.0174 | 0.0256 | 0.0467 | 0.0374 |
| | | 9th | 0.0472 | 0.0716 | 0.0101 | 0.0256 | 0.0350 | 0.0232 |
| | | 10th | 0.0140 | 0.0921 | 0.0067 | 0.0100 | 0.0462 | 0.0310 |
| | 0.0026% | 1st | 0.1175 | 0.0814 | 0.1189 | 0.1091 | 0.0760 | 0.0853 |
| | | 2nd | 0.1165 | 0.1004 | 0.0989 | 0.1101 | 0.0867 | 0.0916 |
| | | 3rd | 0.1072 | 0.1112 | 0.0657 | 0.1038 | 0.0687 | 0.0760 |
| | | 4th | 0.1106 | 0.0814 | 0.0642 | 0.0637 | 0.0667 | 0.0760 |
| | | 5th | 0.1072 | 0.0838 | 0.0500 | 0.0657 | 0.0540 | 0.0794 |
| | | 6th | 0.0955 | 0.0487 | 0.0461 | 0.0681 | 0.0575 | 0.0701 |
| | | 7th | 0.0882 | 0.0604 | 0.0398 | 0.0584 | 0.0653 | 0.0540 |
| | | 8th | 0.0799 | 0.0404 | 0.0315 | 0.0452 | 0.0623 | 0.0438 |
| | | 9th | 0.0813 | 0.0282 | 0.0232 | 0.0281 | 0.0506 | 0.0355 |
| | | 10th | 0.0662 | 0.0248 | 0.0247 | 0.0252 | 0.0350 | 0.0286 |
| Artopine | 10⁻⁶ mol/L | 1st | 0.0228 | 0.0262 | | | | |

TABLE 3

| | Concen- | No. of | Enucleated Organ No./Tension of Contraction (g) | | | | | |
|---|---|---|---|---|---|---|---|---|
| Group | tration | measureing | 1 | 2 | 3 | 4 | 5 | 6 |
| cyanoco- | Control | 1st | 0.1214 | 0.0989 | 0.2649 | 0.2479 | 0.2131 | 0.2136 |
| balamin | 0% | 2nd | 0.1048 | 0.1013 | 0.2439 | 0.2166 | 0.1995 | 0.1609 |
| | | 3rd | 0.1121 | 0.0984 | 0.2205 | 0.1761 | 0.1609 | 0.1482 |
| | | 4th | 0.0936 | 0.0906 | 0.2166 | 0.1766 | 0.1492 | 0.1433 |
| | | 5th | 0.1043 | 0.0759 | 0.1960 | 0.1458 | 0.1384 | 0.1330 |
| | | 6th | 0.0667 | 0.0515 | 0.1233 | 0.1219 | 0.1326 | 0.1247 |
| | | 7th | 0.0726 | 0.0711 | 0.1526 | 0.1219 | 0.1199 | 0.1028 |
| | | 8th | 0.0692 | 0.0598 | 0.1511 | 0.0760 | 0.1272 | 0.0954 |
| | | 9th | 0.0726 | 0.0510 | 0.1316 | 0.0770 | 0.0842 | 0.0876 |
| | | 10th | 0.0658 | 0.0476 | 0.1091 | 0.0413 | 0.0618 | 0.0754 |
| | 0.02% | 1st | 0.2434 | 0.1936 | 0.1136 | 0.2083 | 0.1955 | 0.3015 |
| | | 2nd | 0.2283 | 0.1800 | 0.1039 | 0.2014 | 0.1530 | 0.2531 |
| | | 3rd | 0.2263 | 0.1721 | 0.1009 | 0.1555 | 0.1428 | 0.2453 |
| | | 4th | 0.2229 | 0.1687 | 0.0995 | 0.1409 | 0.1286 | 0.2214 |
| | | 5th | 0.1458 | 0.1594 | 0.0995 | 0.1341 | 0.0720 | 0.2097 |
| | | 6th | 0.1521 | 0.1585 | 0.0941 | 0.1140 | 0.0490 | 0.1828 |
| | | 7th | 0.1428 | 0.1341 | 0.0672 | 0.0852 | 0.0602 | 0.1647 |
| | | 8th | 0.1155 | 0.0594 | 0.0594 | 0.0789 | 0.0319 | 0.1174 |
| | | 9th | 0.1272 | 0.0619 | 0.0619 | 0.0501 | 0.0319 | 0.1086 |
| | | 10th | 0.1526 | 0.1068 | 0.1068 | 0.1160 | 0.0500 | 0.1682 |
| | 0.012% | 1st | 0.1443 | 0.2078 | 0.1067 | 0.2101 | 0.1370 | 0.1809 |
| | | 2nd | 0.1404 | 0.2180 | 0.0857 | 0.2131 | 0.1018 | 0.1809 |
| | | 3rd | 0.1331 | 0.2131 | 0.0872 | 0.1857 | 0.0925 | 0.1575 |
| | | 4th | 0.1267 | 0.1941 | 0.0901 | 0.1730 | 0.0891 | 0.1556 |
| | | 5th | 0.0837 | 0.1687 | 0.0525 | 0.1564 | 0.0574 | 0.1194 |
| | | 6th | 0.0691 | 0.1594 | 0.0579 | 0.1472 | 0.0559 | 0.1267 |
| | | 7th | 0.0710 | 0.1360 | 0.0467 | 0.1193 | 0.0506 | 0.1194 |
| | | 8th | 0.0710 | 0.1160 | 0.0457 | 0.0768 | 0.0466 | 0.1194 |
| | | 9th | 0.0574 | 0.1125 | 0.0467 | 0.1252 | 0.0413 | 0.0833 |
| | | 10th | 0.1189 | 0.2390 | 0.0721 | 0.1965 | 0.0598 | 0.1072 |
| | 0.0072% | 1st | 0.1722 | 0.1546 | 0.1794 | 0.2386 | 0.1712 | 0.2366 |
| | | 2nd | 0.1673 | 0.1433 | 0.1575 | 0.2196 | 0.1179 | 0.2068 |
| | | 3rd | 0.1282 | 0.1380 | 0.1409 | 0.2000 | 0.1072 | 0.1965 |
| | | 4th | 0.1346 | 0.1311 | 0.1272 | 0.1947 | 0.0979 | 0.1824 |
| | | 5th | 0.0955 | 0.1204 | 0.1082 | 0.1785 | 0.0965 | 0.1711 |
| | | 6th | 0.1092 | 0.1233 | 0.0974 | 0.1444 | 0.0960 | 0.1594 |
| | | 7th | 0.0794 | 0.0979 | 0.0925 | 0.1346 | 0.0872 | 0.1404 |
| | | 8th | 0.0647 | 0.0686 | 0.0901 | 0.1273 | 0.0813 | 0.1130 |
| | | 9th | 0.0569 | 0.0921 | 0.0364 | 0.1224 | 0.0759 | 0.0955 |
| | | 10th | 0.1629 | 0.1346 | 0.0691 | 0.2000 | 0.0906 | 0.1584 |
| | 0.0043% | 1st | 0.1116 | 0.1853 | 0.1629 | 0.2034 | 0.1629 | 0.1839 |
| | | 2nd | 0.0955 | 0.1619 | 0.1248 | 0.1711 | 0.1409 | 0.1819 |
| | | 3rd | 0.0584 | 0.1594 | 0.1165 | 0.1262 | 0.1341 | 0.1512 |
| | | 4th | 0.0638 | 0.1482 | 0.0994 | 0.0925 | 0.1238 | 0.1253 |
| | | 5th | 0.0657 | 0.1326 | 0.0809 | 0.0857 | 0.0940 | 0.1077 |
| | | 6th | 0.0603 | 0.1301 | 0.0838 | 0.0930 | 0.0804 | 0.1062 |
| | | 7th | 0.0603 | 0.1272 | 0.0819 | 0.0603 | 0.0745 | 0.0916 |
| | | 8th | 0.0652 | 0.1121 | 0.0726 | 0.0476 | 0.0691 | 0.0852 |
| | | 9th | 0.0691 | 0.1121 | 0.0448 | 0.0549 | 0.0569 | 0.0735 |
| | | 10th | 0.0520 | 0.1150 | 0.1195 | 0.0906 | 0.0975 | 0.0950 |
| | 0.0026% | 1st | 0.1814 | 0.1512 | 0.2185 | 0.1594 | 0.1805 | 0.2034 |
| | | 2nd | 0.1736 | 0.1664 | 0.1912 | 0.1443 | 0.1781 | 0.1922 |
| | | 3rd | 0.1590 | 0.1610 | 0.1658 | 0.1350 | 0.1366 | 0.1561 |
| | | 4th | 0.1487 | 0.1253 | 0.1643 | 0.0984 | 0.1424 | 0.1600 |
| | | 5th | 0.1477 | 0.1288 | 0.1428 | 0.1048 | 0.1234 | 0.1600 |
| | | 6th | 0.1331 | 0.0941 | 0.1448 | 0.0994 | 0.1214 | 0.1248 |
| | | 7th | 0.1316 | 0.1039 | 0.1291 | 0.0930 | 0.1097 | 0.1224 |
| | | 8th | 0.1165 | 0.0716 | 0.1018 | 0.0652 | 0.1146 | 0.1126 |
| | | 9th | 0.1170 | 0.0692 | 0.0608 | 0.0364 | 0.0726 | 0.0731 |
| | | 10th | 0.1023 | 0.0609 | 0.0608 | 0.0388 | 0.0687 | 0.0442 |
| Artopine | 10⁻⁶ mol/L | 1st | 0.0330 | 0.0472 | | | | |

TABLE 4

| | Concen- | No. of | Enucleated Organ No./Tension of Contraction (g) | | | | | |
|---|---|---|---|---|---|---|---|---|
| Group | tration | measureing | 1 | 2 | 3 | 4 | 5 | 6 |
| cyanoco- | Control | 1st | 0.1015 | 0.0674 | 0.0923 | 0.1079 | 0.0611 | 0.0312 |
| balamin | 0% | 2nd | 0.0644 | 0.0537 | 0.0718 | 0.0913 | 0.0679 | 0.0318 |
| | | 3rd | 0.0693 | 0.0376 | 0.0596 | 0.0708 | 0.0640 | 0.0298 |
| | | 4th | 0.0581 | 0.0337 | 0.0801 | 0.0430 | 0.0576 | 0.0259 |
| | | 5th | 0.0639 | 0.0317 | 0.0644 | 0.0703 | 0.0503 | 0.0346 |
| | | 6th | 0.0561 | 0.0395 | 0.0488 | 0.0518 | 0.0513 | 0.0273 |
| | | 7th | 0.0547 | 0.0518 | 0.0239 | 0.0708 | 0.0396 | 0.0181 |
| | | 8th | 0.0533 | 0.0356 | 0.0610 | 0.0401 | 0.0601 | 0.0200 |
| | | 9th | 0.0410 | 0.0356 | 0.0405 | 0.0450 | 0.0278 | 0.0190 |
| | | 10th | 0.0484 | 0.0337 | 0.0219 | 0.0195 | 0.0142 | 0.0171 |
| | 0.02% | 1st | 0.0786 | 0.0537 | 0.0512 | 0.0948 | 0.0361 | 0.0831 |
| | | 2nd | 0.0669 | 0.0254 | 0.0401 | 0.0698 | 0.0190 | 0.0825 |
| | | 3rd | 0.0483 | 0.0288 | 0.0259 | 0.0551 | 0.0201 | 0.0732 |
| | | 4th | 0.0410 | 0.0303 | 0.0318 | 0.0303 | 0.0327 | 0.0659 |
| | | 5th | 0.0269 | 0.0224 | 0.0313 | 0.0552 | 0.0025 | 0.0791 |
| | | 6th | 0.0351 | 0.0215 | 0.0303 | 0.0317 | 0.0083 | 0.0776 |
| | | 7th | 0.0148 | 0.0303 | 0.0190 | 0.0307 | 0.0341 | 0.0634 |
| | | 8th | 0.0103 | 0.0127 | 0.0127 | 0.0327 | 0.0063 | 0.0337 |
| | | 9th | 0.0317 | 0.0142 | 0.0142 | 0.0264 | 0.0127 | 0.0313 |
| | | 10th | 0.0757 | 0.0679 | 0.0679 | 0.0933 | 0.0444 | 0.1324 |
| | 0.012% | 1st | 0.0484 | 0.1128 | 0.0527 | 0.1230 | 0.0640 | 0.0698 |
| | | 2nd | 0.0532 | 0.0981 | 0.0395 | 0.1226 | 0.0385 | 0.0688 |
| | | 3rd | 0.0459 | 0.0976 | 0.0308 | 0.1045 | 0.0390 | 0.0566 |
| | | 4th | 0.0376 | 0.0952 | 0.0259 | 0.1040 | 0.0385 | 0.0372 |
| | | 5th | 0.0307 | 0.0820 | 0.0239 | 0.1006 | 0.0303 | 0.0547 |
| | | 6th | 0.0293 | 0.0737 | 0.0313 | 0.0884 | 0.0302 | 0.0493 |
| | | 7th | 0.0356 | 0.0630 | 0.0318 | 0.0815 | 0.0113 | 0.0464 |
| | | 8th | 0.0293 | 0.0494 | 0.0249 | 0.0346 | 0.0234 | 0.0483 |
| | | 9th | 0.0259 | 0.0546 | 0.0308 | 0.1026 | 0.0230 | 0.0327 |
| | | 10th | 0.0649 | 0.2265 | 0.0581 | 0.1734 | 0.0434 | 0.0810 |
| | 0.0072% | 1st | 0.0997 | 0.0723 | 0.0708 | 0.1792 | 0.0318 | 0.0826 |
| | | 2nd | 0.0752 | 0.0654 | 0.0293 | 0.1216 | 0.0254 | 0.0610 |
| | | 3rd | 0.0869 | 0.0518 | 0.0127 | 0.1045 | 0.0249 | 0.0547 |
| | | 4th | 0.0577 | 0.0468 | 0.0127 | 0.0972 | 0.0180 | 0.0596 |
| | | 5th | 0.0669 | 0.0518 | 0.0191 | 0.0942 | 0.0259 | 0.0551 |
| | | 6th | 0.0625 | 0.0615 | 0.0151 | 0.0855 | 0.0269 | 0.0576 |
| | | 7th | 0.0410 | 0.0351 | 0.0205 | 0.0820 | 0.0205 | 0.0630 |
| | | 8th | 0.0229 | 0.0224 | 0.0166 | 0.1011 | 0.0171 | 0.0366 |
| | | 9th | 0.0410 | 0.0440 | 0.0220 | 0.0903 | 0.0278 | 0.0279 |
| | | 10th | 0.0972 | 0.1045 | 0.0576 | 0.1699 | 0.0386 | 0.1049 |
| | 0.0043% | 1st | 0.0312 | 0.0713 | 0.0962 | 0.0791 | 0.0586 | 0.0904 |
| | | 2nd | 0.0259 | 0.0469 | 0.0708 | 0.0297 | 0.0561 | 0.0884 |
| | | 3rd | 0.0283 | 0.0493 | 0.0844 | 0.0322 | 0.0288 | 0.0796 |
| | | 4th | 0.0235 | 0.0454 | 0.0610 | 0.0312 | 0.0254 | 0.0718 |
| | | 5th | 0.0288 | 0.0391 | 0.0527 | 0.0274 | 0.0151 | 0.0547 |
| | | 6th | 0.0263 | 0.0469 | 0.0578 | 0.0239 | 0.0220 | 0.0605 |
| | | 7th | 0.0253 | 0.0371 | 0.0552 | 0.0229 | 0.0278 | 0.0566 |
| | | 8th | 0.0259 | 0.0274 | 0.0552 | 0.0220 | 0.0224 | 0.0478 |
| | | 9th | 0.0219 | 0.0405 | 0.0347 | 0.0293 | 0.0219 | 0.0503 |
| | | 10th | 0.0380 | 0.0229 | 0.1128 | 0.0806 | 0.0513 | 0.0640 |
| | 0.0026% | 1st | 0.0639 | 0.0698 | 0.0996 | 0.0503 | 0.1045 | 0.1181 |
| | | 2nd | 0.0571 | 0.0660 | 0.0923 | 0.0342 | 0.0914 | 0.1006 |
| | | 3rd | 0.0518 | 0.0498 | 0.1001 | 0.0312 | 0.0679 | 0.0801 |
| | | 4th | 0.0381 | 0.0439 | 0.1001 | 0.0347 | 0.0757 | 0.0840 |
| | | 5th | 0.0405 | 0.0450 | 0.0928 | 0.0391 | 0.0694 | 0.0806 |
| | | 6th | 0.0376 | 0.0454 | 0.0987 | 0.0313 | 0.0639 | 0.0547 |
| | | 7th | 0.0434 | 0.0435 | 0.0893 | 0.0346 | 0.0444 | 0.0684 |
| | | 8th | 0.0366 | 0.0312 | 0.0703 | 0.0200 | 0.0523 | 0.0688 |
| | | 9th | 0.0357 | 0.0410 | 0.0376 | 0.0083 | 0.0220 | 0.0376 |
| | | 10th | 0.0361 | 0.0361 | 0.0361 | 0.0136 | 0.0337 | 0.0156 |
| Artopine | 10⁻⁶ mol/L | 1st | 0.0102 | 0.0210 | | | | |

TABLE 5

| | Concen- | No. of | Enucleated Organ No./Contraction Rate (%) | | | | | |
|---|---|---|---|---|---|---|---|---|
| Group | tration | measureing | 1 | 2 | 3 | 4 | 5 | 6 |
| cyanoco- | Control | 1st | 100.0 | 100.0 | 100.0 | 100.0 | 100.0 | 100.0 |
| balamin | 0% | 2nd | 63.4 | 79.7 | 77.8 | 84.6 | 111.1 | 101.9 |
| | | 3rd | 68.3 | 55.8 | 64.6 | 65.6 | 104.7 | 95.5 |
| | | 4th | 57.2 | 50.0 | 86.8 | 39.9 | 94.3 | 83.0 |
| | | 5th | 63.0 | 47.0 | 69.8 | 65.2 | 82.3 | 110.9 |
| | | 6th | 55.3 | 58.6 | 52.9 | 48.0 | 84.0 | 87.5 |
| | | 7th | 53.9 | 76.9 | 25.9 | 65.6 | 64.8 | 58.0 |
| | | 8th | 52.5 | 52.8 | 66.1 | 37.2 | 98.4 | 64.1 |
| | | 9th | 40.4 | 52.8 | 43.9 | 41.7 | 45.5 | 60.9 |
| | | 10th | 47.7 | 50.0 | 23.7 | 18.1 | 23.2 | 54.8 |
| | 0.02% | 1st | 100.0 | 100.0 | 100.0 | 100.0 | 100.0 | 100.0 |
| | | 2nd | 85.1 | 47.3 | 78.3 | 73.6 | 52.6 | 99.3 |
| | | 3rd | 61.5 | 53.6 | 50.6 | 58.1 | 55.7 | 88.1 |
| | | 4th | 52.2 | 56.4 | 62.1 | 32.0 | 90.6 | 79.3 |
| | | 5th | 34.2 | 41.7 | 61.1 | 58.2 | 6.9 | 95.2 |
| | | 6th | 44.7 | 40.0 | 59.2 | 33.4 | 23.0 | 93.4 |
| | | 7th | 18.8 | 56.4 | 37.1 | 32.4 | 94.5 | 76.3 |
| | | 8th | 13.1 | 23.6 | 24.8 | 34.5 | 17.5 | 40.6 |
| | | 9th | 40.3 | 26.4 | 27.7 | 27.8 | 35.2 | 37.7 |
| | | 10th | 96.3 | 126.4 | 132.6 | 98.4 | 123.0 | 159.3 |
| | 0.012% | 1st | 100.0 | 100.0 | 100.0 | 100.0 | 100.0 | 100.0 |
| | | 2nd | 109.9 | 87.0 | 75.0 | 99.7 | 60.2 | 98.6 |
| | | 3rd | 94.8 | 86.5 | 58.4 | 85.0 | 60.9 | 81.1 |
| | | 4th | 77.7 | 84.4 | 49.1 | 84.6 | 60.2 | 53.3 |
| | | 5th | 63.4 | 72.7 | 45.4 | 81.8 | 47.3 | 78.4 |
| | | 6th | 60.5 | 65.3 | 59.4 | 71.9 | 47.2 | 70.6 |
| | | 7th | 73.6 | 55.9 | 60.3 | 66.3 | 17.7 | 66.5 |
| | | 8th | 60.5 | 43.8 | 47.2 | 28.1 | 36.6 | 69.2 |
| | | 9th | 53.5 | 48.4 | 58.4 | 83.4 | 35.9 | 46.8 |
| | | 10th | 134.1 | 200.8 | 110.2 | 141.0 | 67.8 | 116.0 |
| | 0.0072% | 1st | 100.0 | 100.0 | 100.0 | 100.0 | 100.0 | 100.0 |
| | | 2nd | 75.4 | 90.5 | 41.4 | 67.9 | 79.9 | 73.8 |
| | | 3rd | 87.2 | 71.6 | 17.9 | 58.3 | 78.3 | 66.2 |
| | | 4th | 57.9 | 64.7 | 17.9 | 54.2 | 56.6 | 72.2 |
| | | 5th | 67.1 | 71.6 | 27.0 | 52.6 | 81.4 | 66.7 |
| | | 6th | 62.7 | 85.1 | 21.3 | 47.7 | 84.6 | 69.7 |
| | | 7th | 41.1 | 48.5 | 29.0 | 45.8 | 64.5 | 76.3 |
| | | 8th | 23.0 | 31.0 | 23.4 | 56.4 | 53.8 | 44.3 |
| | | 9th | 41.1 | 60.9 | 31.1 | 50.4 | 87.4 | 33.8 |
| | | 10th | 97.5 | 144.5 | 81.4 | 94.8 | 121.4 | 127.0 |
| | 0.0043% | 1st | 100.0 | 100.0 | 100.0 | 100.0 | 100.0 | 100.0 |
| | | 2nd | 83.0 | 65.8 | 73.6 | 37.5 | 95.7 | 97.8 |
| | | 3rd | 90.7 | 69.1 | 87.7 | 40.7 | 49.1 | 88.1 |
| | | 4th | 75.3 | 63.7 | 63.4 | 39.4 | 43.3 | 79.4 |
| | | 5th | 92.3 | 54.8 | 54.8 | 34.6 | 25.8 | 60.5 |
| | | 6th | 84.3 | 65.8 | 60.1 | 30.2 | 37.5 | 66.9 |
| | | 7th | 81.1 | 52.0 | 57.4 | 29.0 | 47.4 | 62.6 |
| | | 8th | 83.0 | 38.4 | 57.4 | 27.8 | 38.2 | 52.9 |
| | | 9th | 70.2 | 56.8 | 36.1 | 37.0 | 37.4 | 55.6 |
| | | 10th | 121.8 | 32.1 | 117.3 | 101.9 | 87.5 | 70.8 |
| | 0.0026% | 1st | 100.0 | 100.0 | 100.0 | 100.0 | 100.0 | 100.0 |
| | | 2nd | 89.4 | 94.6 | 92.7 | 68.0 | 87.5 | 85.2 |
| | | 3rd | 81.1 | 71.3 | 100.5 | 62.0 | 65.0 | 67.8 |
| | | 4th | 59.6 | 62.9 | 100.5 | 69.0 | 72.4 | 71.1 |
| | | 5th | 63.4 | 64.5 | 93.2 | 77.7 | 66.4 | 68.2 |
| | | 6th | 58.8 | 65.0 | 99.1 | 62.2 | 61.1 | 46.3 |
| | | 7th | 67.9 | 62.3 | 89.7 | 68.8 | 42.5 | 57.9 |
| | | 8th | 57.3 | 44.7 | 70.6 | 39.8 | 50.0 | 58.3 |
| | | 9th | 55.9 | 58.7 | 37.8 | 16.5 | 21.1 | 31.8 |
| | | 10th | 56.5 | 51.7 | 36.2 | 27.0 | 32.2 | 13.2 |
| Artopine | 10⁻⁶ mol/L | 1st | — | — | | | | |

TABLE 6

| | Enucleated Organ No./ | | | Inhibition |
|---|---|---|---|---|
| Concen- | Tension of Contraction (g) | | Average ± standard | Value |
| tration | 1 | 2 | deviation | (%) |
| Control | 0.1487 | 0.1693 | 0.1590 ± 0.0146 | — |
| 10⁻⁶ mol/L | 0.0228 | 0.0262 | 0.0245 ± 0.0024 | 84.6 |

TABLE 7

| | Enucleated Organ No./ | | | Inhibition |
|---|---|---|---|---|
| Concen- | Tension of Contraction (g) | | Average ± standard | Value |
| tration | 1 | 2 | deviation | (%) |
| Control | 0.1829 | 0.2630 | 0.2230 ± 0.0566 | — |
| 10⁻⁶ mol/L | 0.0330 | 0.0472 | 0.0401 ± 0.0100 | 82.0 |

TABLE 8

| | Enucleated Organ No./ | | | Inhibition |
|---|---|---|---|---|
| Concen- | Tension of Contraction (g) | | Average ± standard | Value |
| tration | 1 | 2 | deviation | (%) |
| Control | 0.0342 | 0.0937 | 0.0640 ± 0.0421 | — |
| 10⁻⁶ mol/L | 0.0102 | 0.0210 | 0.0156 ± 0.0076 | 75.6 |

As seen in Tables 1-5 and in FIG. 1, fatiguing of the ciliary muscle occurs upon stimulation by acetylcholine. The contraction rate decreases as repeated stimulations by acetylcholine are applied. It is evident from Tables 6-8 that the contraction occurs via the muscarinic receptor, since contraction of the ciliary muscle is elicited by acetylcholine and inhibited by atropine.

### EXAMPLE 3

### Preliminary Test

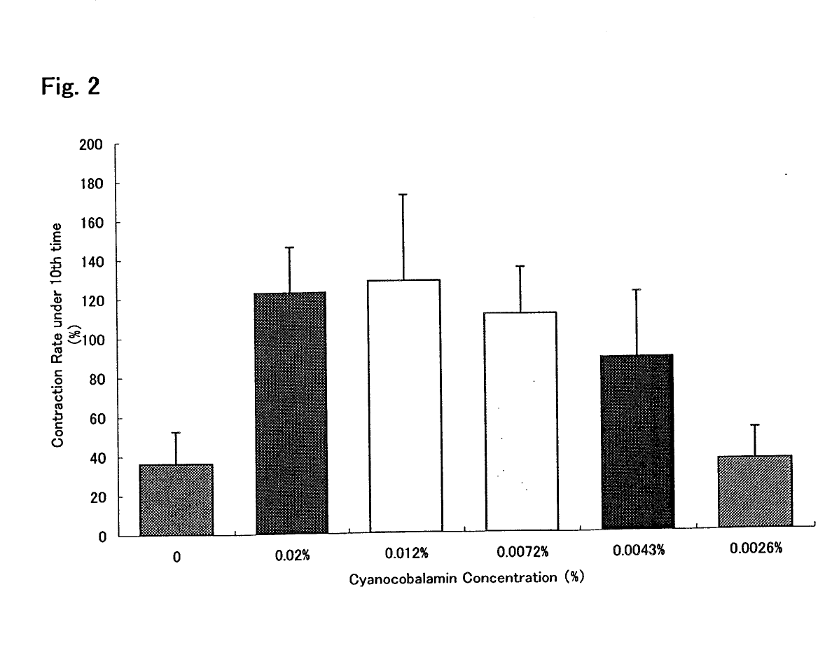

Before the 10th(and last) stimulation, the solution in the Magnus tube was replaced with the Krebs-Henseleit solution containing cyanocobalamin at a concentration of 0, 0.02, 0.012, 0.0072, 0.0043 and 0.0026%, and then the 10th stimulation was applied to the sample. Cyanocobalamin was used in the above preliminary test, because it is the active ingredient of the test formulation used in the following test. The results are shown in Tables 1-5 and FIGS. 1 and 2.

The Krebs-Henseleit solution, containing cyanocobalamin, was prepared as follows. A-solution, B-solution, C-solution, distilled water, 10× cyanocobalamin stock solution and D-solution were mixed at a ratio of 1:1:1:5:1:1 in the following order. First A-solution, B-solution and C-solution were mixed, to which was added distilled water. The 10-fold concentrated cyanocobalamin solution was then added, followed by the addition of D-solution. The solution thus prepared was shielded from light and used after heating at 37 degrees C.

As shown in Tables 1-5 and FIG. 1 and FIG. 2, no tension increase was observed with 0.0026% cyanocobalamin upon the 10th stimulation, compared with the previous stimulation. At 0.0043%, however, the contraction was improved. Tension recovery increased with increasing concentration. The anti-fatiguing effect of cyanocobalamin appeared to reach a plateau at 0.012%. As shown in Table 2, cyanocobalamin showed no effect on the baseline tension. Based on the above results, the concentration of cyanocobalamin for the final test was set at 0.012%.

### EXAMPLE 4

### Final Test

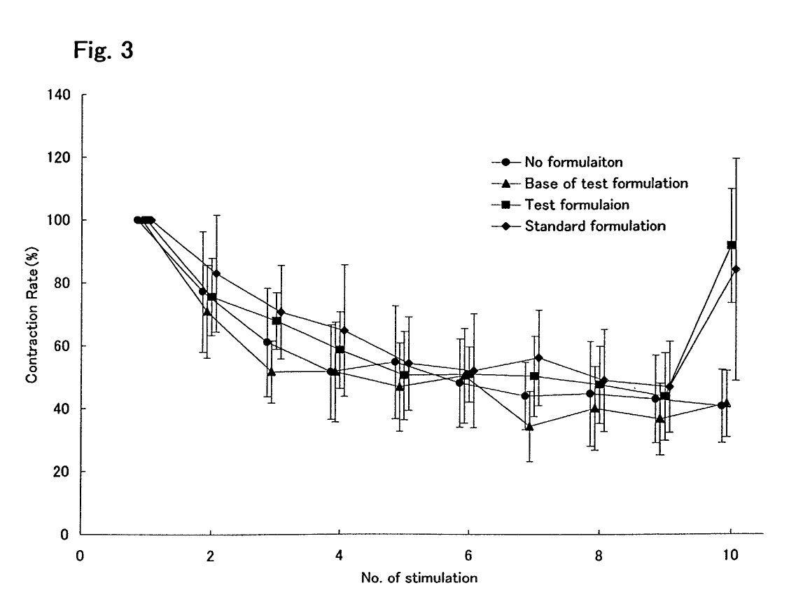

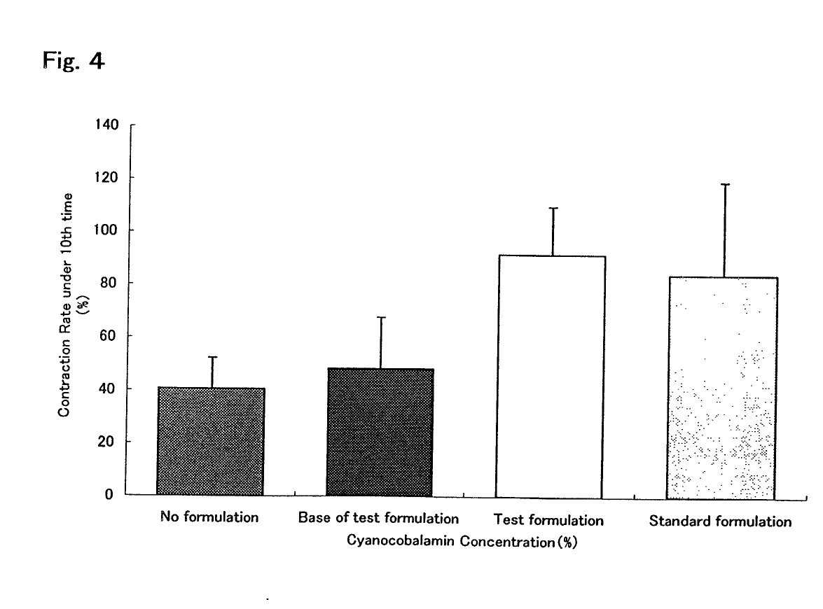

The same protocol as used in EXAMPLE 3 was carried out, but using the following test formulation: TP263 (made by Toyo Pharma Kabushiki Kaisha), corresponding to 0.02% cyanocobalamin eye drop; and the following standard formulation: Sancoba eye drop 0.02% (made by Santen Pharmaceutical Co., Ltd.), instead of cyanocobalamin. The results were evaluated using the Turkey multiple comparative method. The results are shown in Table 9 and FIG. 3 and FIG. 4.

TABLE 9

| Existance of | No. of | Average ± | |
|---|---|---|---|
| formulation | stimulaiton | standard deviation | Tukey multiple comparison |
| None | 2nd | 77.3 ± 19.2 76.3 ± 17.6 75.7 ± 12.3 83.1 ± 18.6 | 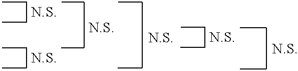 |
| None | 3rd | 61.2 ± 17.3 55.1 ± 12.1 68.0 ± 9.0 70.8 ± 14.9 | 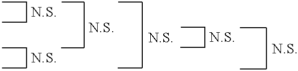 |
| None | 4th | 51.6 ± 15.1 56.6 ± 17.7 58.6 ± 12.2 64.8 ± 21.0 | 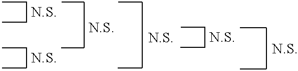 |
| None | 5th | 54.7 ± 18.0 50.4 ± 13.8 50.5 ± 14.1 54.3 ± 14.9 | 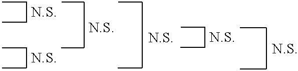 |
| None | 6th | 48.1 ± 14.1 55.4 ± 16.6 50.9 ± 8.8 52.0 ± 18.2 | 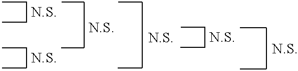 |
| None | 7th | 44.0 ± 10.7 39.6 ± 15.6 50.3 ± 12.8 56.2 ± 15.2 | 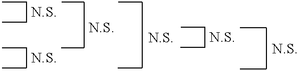 |
| None | 8th | 44.7 ± 16.7 43.8 ± 13.6 47.6 ± 12.3 49.0 ± 16.3 | 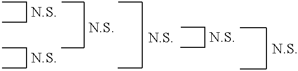 |
| None | 9th | 43.0 ± 14.0 41.7 ± 14.4 43.8 ± 14.0 46.9 ± 14.5 | 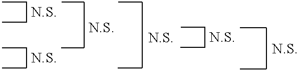 |
| No formulation Base of test formulation Test formulation Standard formulation | 10th | 40.7 ± 11.7 48.4 ± 19.3 91.9 ± 18.2 84.2 ± 35.4 | 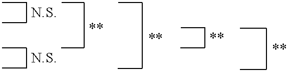 |

**P<0.01,
*P<0.05,
N.S.:no significant

As seen by the results in Table 9 and FIG. 3 and FIG. 4, the test formulation had an anti-fatiguing effect on the ciliary muscle. The effect of the test formulation was higher than the vehicle formulation without cyanocobalamin. Comparison of the test formulation and the standard formulation shows that they have a similar level of anti-fatiguing effect on the ciliary muscle.

As can be seen from the foregoing, the experimental model of the present invention is advantageous in that it replicates asthenopia occurring in vivo, and it gives a substantially stable decrease in the tension of muscular contraction which enables persons of ordinary skill in the art to evaluate, in vitro, the therapeutic effect of a potential medicine on asthenopia quantitatively. The known in vitro model described in Japan patent publication No. H07-133225, published, may be useful as an experimental model for evaluating or screening a medicine which shows an antagonistic effect on the transient contraction of ciliary muscle induced by chemical stimulation, but not for evaluating the therapeutic effect on asthenopia, because asthenopia is not necessarily ascribed to such transient ciliary muscle contractions. In addition, that known in vitro model simply provides a screening method to evaluate the preventive effect of a medicine on muscle contraction by contacting or treating the ciliary muscle with the medicine before inducing the ciliary muscle contraction. In contrast, the experimental model and method of the present invention involves repeated contractions of ciliary muscle which give a stable decrease in the tension of muscular contraction, thus replicating the fatiguing of ciliary muscle which occurs in asthenopia, and hence this invention is much more advantageous for use in evaluating the therapeutic effect of a medicine against asthenopia. According to the present invention, the therapeutic effect of the medicine is evaluated after the fatigue of the ciliary muscle is induced. Thus, the same sample of ciliary muscle can be used to test multiple medicines, which enables persons engaged in this art to compare various medicines without the influence of specimen-to-specimen variations.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An experimental model for evaluating the effect of a medicine against asthenopia, wherein said asthenopia is caused by inducing repeated contraction in vitro of ciliary muscle from a non-human animal until said ciliary muscle shows a substantially stable decrease in the tension of muscular contraction.

2. The experimental model of claim 1, wherein said asthenopia is accommodative asthenopia.

3. The experimental model of claim 1, wherein the ciliary muscle has been derived from a non-human mammal or fowl.

4. The experimental model of claim 2, wherein the ciliary muscle has been derived from a non-human mammal or fowl.

5. The experimental model of claim 3, wherein the ciliary muscle has been enucleated from a non-human mammal.

6. The experimental model of claim 4, wherein the ciliary muscle has been enucleated from a non-human mammal.

7. The experimental model of claim 1, wherein the ciliary muscle is contracted a plurality of times by the use of smooth muscle contraction-inducing means.

8. The experimental model of claim 7, wherein the smooth muscle contraction-inducing means comprise a chemical stimulant.

9. The experimental model of claim 8, wherein the chemical stimulant is selected from the group consisting of acetylcholine, serotonin, histamine, muscarine, nicotine and endothelin.

10. The experimental model of claim 7, wherein the smooth muscle contraction-inducting means comprises an electrical stimulant.

11. The experimental model of claim 1, wherein the contraction of ciliary muscle is repeated at least three times to give a substantially stable decrease in the tension of muscular contraction.

12. The experimental model of claim 1, wherein said ciliary muscle shows a decrease of 50±30% in the tension of muscular contraction.

13. The experimental model of claim 1, wherein said ciliary muscle shows a decrease of 50±20% in the tension of muscular contraction.

14. The experimental model claim 1, wherein said ciliary muscle shows a decrease of 50±10% in the tension of muscular contraction.

15. A method of preparing an in vitro experimental model for evaluating the effect of a medicine against asthenopia, which comprises the step of inducing repeated contractions of ciliary muscle derived from a non-human animal until said ciliary muscle shows a substantially stable decrease in the tension of muscular contraction.

16. A method for evaluating a medicine against asthenopia, comprising the steps of contacting the ciliary muscle from a non-human animal in the experimental model of claim 1 with said medicine, and measuring the effect of said medicine on the contraction of said ciliary muscle.

17. The method of claim 16, wherein the effect of the medicine is evaluated by comparing the decrease in tension of muscular contraction before and after contacting with the medicine.

18. The method claimed of claim 16, carried out with use of a Magnus apparatus.
